(19) United States
(12) Patent Application Publication
(10) Pub. No.: US 2008/0254138 A1
(43) Pub. Date: Oct. 16, 2008

(54) EXTRACELLULAR MATRIX TO RECRUIT STEM CELLS TO A SITE OF MALIGNANCY

(76) Inventor: LEIGH H. FIRESTONE, Berkeley, CA (US)

Correspondence Address: LEIGH HUNT FIRESTONE, 7100 NORFOLK ROAD, BERKELEY, CA, 94705, US

(21) Appl. No.: 11/925,907

(22) Filed: Oct. 27, 2007

Related U.S. Application Data
(63) Continuation-in-part of application No. 11/708,231, filed on Feb. 20, 2007.
Application No. 11/708,231 is a continuation-in-part of application No. 11/865,023, filed on Sep. 30, 2007.
Application No. 11/865,023 is a continuation-in-part of application No. 11/876,963, filed on Oct. 23, 2007.
(60) Provisional application No. 60/775,913, filed on Feb. 22, 2006.

(30) Foreign Application Priority Data
Feb. 22, 2007 (US) 2007/004332

(51) Int. Cl. A61K 35/37; A61K 35/36; A61K 35/12; A61P 35/00; A61K 35/38

(52) U.S. Cl. 424/551; 424/520; 424/574

(57) ABSTRACT

The invention is to a method of recruiting stem cells to a site of malignancy by contacting the site with exogenous mammalian extracellular matrix in the form of a sheet article, or a composition comprising particulate extracellular matrix, or emulsion or gel extracellular matrix.

### CROSS-REFERENCES TO RELATED APPLICATIONS

The present application is a continuation in part to application U.S. Ser. No. 11/708,231 filed Feb. 20, 2007 which draws priority from U.S. provisional application 60/775,913 filed Feb. 22, 2006. The present application also claims priority from PCT application PCT/US2007/004332 filed Feb. 21, 2007. The present application is also a continuation in part of U.S. Ser. No. 11/865,023 filed Sep. 30, 2007. The present application is also a continuation in part of U.S. Ser. No. 11/876963 filed Oct. 23, 2007. All related applications are specifically incorporated by reference in their entirety.

### FIELD OF THE INVENTION

The invention is to a compositions (particulate and emulsion or gel) and articles (sheets or laminates of sheets) of extracellular matrix to be used to recruit stem cells to a site of malignancy in a mammal in order to treat that malignancy.

### BACKGROUND OF THE INVENTION

Carcinoma is the term for abnormally proliferating and poorly differentiating epithelial cells in mammals. Many types of carcinomas exist, including the very prevalent adenocarcinoma, which describes cancer of the epithelial surfaces of glandular tissues. Breast, colon, lung, thyroid, prostate, stomach, pancreatic, cervical, and ovarian cancers are all examples of adenocarcinomas, although some smaller subsets of cancers in these organs can be other than epithelial cancers. In addition, carcinomas can originate in the bladder, uteris, kidney, lung, and skin. This list is not exhaustive.

Types of normal healthy epithelium include simply squamous cells, simple cuboidal cells, simple columnar cells, stratified squamous cells, stratified cuboidal cells, pseudosratified columnar cells and transitional cells. Depending on the organ or tissue, the epithelial cells will take on their appropriate character within these categories.

Cancers of epithelial origin are lesions or groups of abnormally proliferating cells that form solid tumors. Cells within tumors eventually break off from their site of origin and metastize or move to other tissues. Those other tissues can be both epithelial and non-epithelial in character.

Tumors are graded or staged depending on their level of differentiation and localization to the site of tumor origin. For example, Stage I ovarian tumors are confined to one or both ovaries. Stage II ovarian is ovarian cancer that has spread to pelvic organs, but not to abdominal organs. Stage III is ovarian cancer that has spread to abdominal organs. Stage IV is ovarian cancer that has spread outside to distant sites, for example the lung, brain, or lymph nodes in the neck. Recurrent ovarian cancer is cancer that has recurred after completion of a treatment. Within these stages there are subcategories that are identified based on tumor size, node involvement and metastatic status. Thus a tumor can be a T2a, which describes a tumor that has spread and attached to the uterus, where as a T2b tumor describes a tumor that has in addition spread to other pelvic tissues, but with no cancer cells in the ascites or peritoneum, and so on. In addition to tumor staging, epithelial tumors can also be graded. Grade 1 is the least malignant with well-differentiated cells, grade 2 is intermediate with moderately differentiated cells, and Grade 3 if the most malignant with poorly differentiated cells. Low grade tumors grow more slowly and have a better prognosis for survival.

Further continuing with the ovarian model for exemplary purposes, treatment of epithelial cancers usually involve a first step especially if the tumor is Stage 1A or Stage 1B. Surgery can include hysterectomy (removal of the uterus), bilateral slpingectomy (removal of both fallopian tubes), biolateral oophorectomy (removal of both ovaries), and omenectomy (removal of part of the ornamentum, which if fatty tissue from the upper part of the abdominal cavity near the stomach and intestines). Pelvic and aortic lymph nodes may be sampled and the linings of the pelvis and abdominal cavities can be biopsied to determine if the cancer has spread. If the tumor is deemed a grade 1 or 2, meaning that the cells have some similarities to normal cells, surgery alone may be a cure for the patient. Even if the cancer has not spread, but turns out to be a grade 2 or 3 cell, then chemotherapy may be recommended after surgery. If the cancer is Stage II, then the standard protocol is to “debulk” the tumor in the pelvis as much as possible, which means to remove as much tumor tissue as can be located and safely removed. The protocol for Stage III and Stage IV are the same as for Stage II, with initial surgical treatment followed by chemotherapy. In some Stage III and Stage IV situations, the surgeon will administer chemotherapy directly to the tissues intraperitoneally, and patients have been found to survive longer with this aggressive approach. Follow-up surgery can include laparoscopy to determine if any tumors have regrown in the area of original surgery. Tumor reccurrence can also be deduced from blood marker tests, palpation, positron emission tomography, CT scans, and magnetic resonance imaging (MRI).

Modern treatments for cancer include surgery to remove the tumor, hormone therapy, radiation, chemotherapy and immunotherapy. As it is often an incurable disease, many patients also desperately experiment with unproven and unapproved therapies of questionable clinical efficacy in an effort to save their lives. Modern understanding of the mechanism of action of cancer provides direction for new experimental drugs and includes observations of an increase in angiogenesis at the tumor site, development amongst the tumor cells of cancer specific signal transduction pathways, and genetic characteristics that identify over or under expressed genes in cancer populations and provide direction for designs for protein, nucleic acid, or small molecules actors to target the cancer cells.

Cancer usually forms solid tumors, such as adenocarcinoma and other solid tumor cancers. In addition there are cancers that involve a fluid system in the body, cancers such leukemia and lymphoma. Different types of cancer behave differently with different growth rates, cell characteristics, markers to identify the cancer cells, responsiveness to different types of treatment, and metastatic potential and tendencies.

Because cancer begins at the microscopic cellular level, the first signs of malignant growth are nearly impossible to detect without special tests. One of the most insidious aspects of cancer is the way it grows. As the tumor outgrows the original organ, pieces of the malignant tissue often break off or metastasize and enter the blood stream or lymph system. The migrating cancer cells then attach themselves to different organs and form new tumors. Survival rates for cancer increase if the primary tumor is found early and excised. However, even with a clean excision where the surgeon believes all of the tumor has been removed, a few cells can remain at the site, and regrow another tumor. In addition, some tumor excisions are performed when the tumor has probably already metastasized, and therefore it is nearly impossible to retrieve all the cells of the original tumor. There are also theories that cutting the tumor spreads one or more cancer cells into the blood stream which precipitates metastasis regardless of whether a clean margin is attained for the tumor resection. Thus, if it were possible to treat solid tumors without resection, that would be a great medical benefit.

It would be a great triumph for medicine and world health if a composition could be developed that lowered the risk of death by cancer and provided an effective treatment.

### SUMMARY OF THE INVENTION

The invention is to a method of recruiting an endogenous stem cell to a site of abnormally proliferating cells in mammalian tissue comprising: a) locating a site of an abnormally proliferating cell in a tissue type of a mammal, b) contacting said site with a composition comprising exogenous native mammalian extracellular matrix, and c) detecting recruitment of an endogenous stem cell to said site.

The abnormally proliferating cell can be an epithelial cell.

The tissue type can be epithelial tissue.

The extracellular matrix can be liquid or semi-solid (e.g. fluidized ECM, or emulsion or gel ECM). The extracellular matrix can be a particulate.

The extracellular matrix can be a sheet.

The extracellular matrix can be porcine, bovine, or human.

The extracellular matrix can be small intestine submucosa, liver basement membrane, urinary bladder submucosa, or stomach submucosa.

The abnormally proliferating cell can be malignant.

The method can further comprise a step of disturbing said abnormally proliferating cell at said site before contacting the site with the composition.

The disturbing step can comprises removal of all or part of said site of abnormally proliferating cells.

The stem cell can be an adult stem cell.

The mammalian tissue can be pancreatic tissue, liver tissue, breast tissue, ovarian tissue, or lung tissue.

### DETAILED DESCRIPTION OF THE INVENTION

The invention is use of exogenous native extracellular matrix to treat a site of malignancy. The placement of the extracellular matrix at the site recruits endogenous adult stem cells to the site, which prevents recurrence of the malignancy when a tumor has been excised at the site. The invention employs compositions or articles of healthy soft tissue extracellular matrix. Soft tissue extracellular matrix is extracellular matrix derived from soft tissue (epithelial tissues) of mammals. In the presence of healthy exogenous mammalian extracellular matrix placed at a site of malignancy, stem cells are recruited to the site which redirects the course of the malignant development in the tissue. As a result, malignant cancer cells are inhibited from their abnormal cell growth and eventually die. The invention describes contacting an abnormally proliferating cell with sufficient soft tissue extracellular matrix to inhibit the abnormal cell proliferation. The invention claims recruitment of stem cells to a site of malignancy. The stem cells work within the extracellular matrix to form new tissue at the site, healthy tissue that inhibits cancer cell growth, and reduces a likelihood of recurrence of cancer growth at the site of the original tumor. How this inhibition occurs, and whether it is by a mechanism such as making the cells undergo apoptosis, undergo cell cycle arrest, or some combination of cellular events that result in either the death of the cancer cells or an arrest of their proliferation, has not been determined.

For the purposes of this invention, that a malignant cell or group of malignant cells fails to continue to proliferate is sufficient understanding for use of the compositions and methods of the invention towards treatment of an individual afflicted with the presence of such malignant and abnormally proliferating cells. The contact made between the extracellular matrix and the abnormally proliferating cell is made in vivo, in the person or animal afflicted with the abnormal cell proliferation disorder. The contact can be made with the abnormally proliferating cell in the tissue that the cell is growing. For example, a surgeon can identify a tumor in a patient and contact the tumor with sufficient extracellular matrix to cover and surround the tumor or aggregrate of cancer cells thereby providing a material to the site that will recruit stem cells to the site of malignancy and alter the course of the malignancy.

Alternatively, a group of such abnormally proliferating cells can be removed and the extracellular matrix composition can be applied at the site of tumor removal, to prevent tumor recurrence by recruiting endogenous adult stem cells to the site and regenerating healthy tissue. Thus the extracellular matrix composition can be applied to tissue after surgical resection of a tumor, before closing the site. To accomplish good contact with the tissue at the site, emulsified, injectable, foam, gel, liquid, glue, paste, small piece, patch, strip, pellet, plug, strand, weave, spray, paint, cream or any malleable, tissue-attachable form of the extracellular matrix material is used. Conceivably, small pieces, small patches, plugs, strips, pellets, or strands of extracellular matrix material can be attached at the site, or placed there and will work to regenerate healthy tissue at the site of tumor resection. In general, any malleable or appropriate tissue-attachable form of extracellular matrix material that can also contact the abnormally growing cells attached to the tissue can be used at the site, the form primarily being determined by the nature of the tissue, the nature of the cancer cells being targeted for growth inhibition, the nature of the resection, and the anticipated needs for healing and tissue regeneration at the site.

The invention is to includes a method of facilitating optimal wound healing after tumor removal. Patients having tumors growing in soft tissue undergo surgery to remove these tumors. Depending on the size and location of the tumors a space is left at the tumor site, where removal of the tumor and some surrounding tissue leaves a cavity. Normally this space is not filled with anything, but the surgical site is closed and the patient's wound allowed to heal. The invention provides a method by which a therapeutic composition comprising extracellular matrix is applied to the surgical site, preferably filling the cavity, and then the site is closed. Provision of a therapeutically effective amount of extracellular matrix at the site facilitates the wound to heal with some or complete regeneration of the removed tissue, and with reduced or absent formation of scar tissue. This creates an environment that reduces the reformation and recurrence of malignant growth at the site of the original tumor excision.

The patient, individual, subject, human, or animal is afflicted with a cell proliferation disorder. These terms identifying the person or object of the treatment are all synonymous for the purposes of identifying the living entity afflicted with the cell proliferation disorder, or in other words the person carrying the one or more malignant cells.

The cell proliferation disorders that can be addressed with the compositions and method of the invention are generally some form or abnormally growing cells, particularly abnormally proliferating cells, especially malignant cells, and including generally all types of cell proliferation disorders. It is acknowledged that the main focus of abnormal growth is abnormal proliferation, the mechanism by which malignant cancer cells overtake the body they live in.

The extracellular matrix is from a mammalian source. Accordingly, any mammal can be the source of deriving any form of extracellular matrix for the purpose of making it into a composition that is then used to contact abnormally proliferating cells in the afflicted individual. Such available mammals include humans, horses, monkeys, cows, pigs, sheep, rabbits, rodents, and generally any otherwise healthy mammal. It is also possible that other animals, including fish and birds may provide extracellular matrix of sufficient quality for use in the compositions. The extracellular matrix can be natural. Examples of natural extracellular matrix include extracellular matrix derived from a mammal, such as small intestine submucosa (SIS), urinary bladder submucosa (UBS), liver basement membrane (LBM), or other such tissues derived from mammalian animal tissue. It may be possible that the natural extracellular matrix can be plant derived, such as collagen or glycosaminoglycans from plants. Additives can be mixed into the extracellular matrix. Additives can include such molecules as immunotherapeutic molecules, cells, anticancer agents, nucleic acids, peptides, polypeptides and proteins.

Extracellular matrix is a broad term meant to encompass any protein matrix residing outside cells in animal tissue. There has been much work using many different forms of extracellular matrix in tissue regeneration for all types of wounds and damage to tissue. Accordingly there have been many different and specific definitions and descriptions that pertain to various types of extracellular matrices from various different tissues. In addition, many extracellular matrices have multiple layers, and these layers have various terms, including basement membrane, submucosa, lamina propia, epithelial layers and the like. This list of terms that identify parts of extracellular matrix is not meant to be exhaustive, but merely illustrates the point that there are many sub-species of extracellular matrix that can include as little as a single layer or a couple of layers of a larger extracellular matrix material. The invention contemplates all of these matrix layers, terms, and definitions from the broad to the narrow. The invention appreciates the usefulness of any and all of these matrices in facilitating the purpose of the invention, and therefore does not mean to limit the compositions or the methods of the invention to any particular class or subclass or definitional niche of this very broad category called extracellular matrix. Further the extracellular matrices of the invention comprise extracellular matrix proteins. The various combinations, amounts, qualities, and types of proteins that make up the extracellular matrix protein for each type of extracellular matrix are contemplated by the invention. In many cases the combinations of extracellular matrix protein of a given extracellular matrix defines the matrix, including its polymer structure made up of polymerized proteins and its functional capacity for tissue regeneration, abnormal cell growth inhibition, and cell-cell, protein-cell, and protein-protein communications.

Where a tumor has been identified in the patient, the tumor can be addressed by contacting the tumor with an aliquot of extracellular matrix sufficient to cover the tumor and contact all the abnormally growing cells possible. The tumor may also be resected and the space left by the removed tumor can be filled with extracellular matrix. The tumor growth can include, for example, any tumor stages, such as tumor growth as a solid tumor, carcinoma, sarcoma, lymphoma, hyperplasia, dysplasia, neoplasia, abnormal cell proliferation, restenosis, malignant cell growth, metastatic cell, cancer cell, or precancer cell. As stated earlier, the composition can also be placed at a site of abnormal cell growth, or in contact with a population of cancer or precancer cells. Placing extracellular matrix at the site of abnormal cell growth can cause an interruption of the abnormal cell growth, and a remodeling of the unhealthy tissue to new healthy tissue. Placing extracellular matrix at a site having cancer or precancer cells can serve to eliminate the cancer cells from the local tissue environment and so eliminate the cells and their potential for tumor formation in the body. Placing extracellular matrix in the tumor space can ensure that the tumor will not recur at the site of removal, and will additionally help to heal the tissue so that scarring and disfigurement is limited.

Means of placing the extracellular matrix at the site in the body where a tumor has been resected, or cancer cells are believed to exist, can be accomplished by surgically opening the site and accessing the tissue directly, or by percutanous or other minimally invasive access to the site of cancer or the site where cancer is believed to be. Direct injection of the extracellular matrix at the site either of abnormal cell proliferation or tumor resection may be preferred. A catheter may be able to resect the tumor or lesion of abnormal cells and afterwards apply a coating of extracellular matrix in an injectable, emulsion or spray, for example. Solid pieces of extracellular matrix can be sutured, stapled, glued, or otherwise attached at a site. Plugs, pellets or other pieces of extracellular matrix can be placed or attached at the site using an appropriate attachment means, such a glue or suture.

While the invention is not limited to theories about how it might work, it is postulated that after tumor resection, if the body is provided with an opportunity to regenerate healthy tissue, by replacing the excised tissue, the chance of tumor recurrence would decrease. The signal for healthy tissue regeneration would come from the extracellular matrix material as it recruits stem cells, proteins, and other biological actors to grow new tissue at the site. Eventually, the original extracellular matrix material placed at the site is completely replaced by new healthy tissue. Normally, when tissue and tumor are resected the site is closed. Any lingering cancer cells may regrow into a second tumor. In addition, the resected healthy tissue is left to heal essentially unaided by any external or exogenous assistance.

Placement of some form of extracellular matrix at the site of resection will optimize chances of cancer-free recovery at the site, and reduce scarring or other trauma from the wound caused by the tumor resection. Where progression of cancer is often measured by the tumor-load or amount of cancerous cells estimated to be in the body, death by cancer is anticipated when the tumor-load reaches a percentage that overwhelms the healthy cells in the body. Extracellular matrix may be able to send healing signals to the afflicted organ tissue to help it fight the cancer growth and reorganize its local signals to have healthy tissue grow preferentially in favor of unhealthy cancerous or precancerous tissue. Abnormal cell growth can be any form of cell proliferation, including, for example restenotic cell growth, such as the proliferative cells seen in arterial plaque lesions in blood vessels. Any means that can reduce the return or presence of cancer cells in the body will by definition prolong the life of the cancer patient.

The invention is also a composition of extracellular matrix material applied to a site having a population of cancer or precancer cells for the purpose of eliminating the cancer or precancer cells from the site, without first resecting a tumor or lesion of the cells. The cancer or precancer cells may be resected or they may remain at the site.

The extracellular matrix can be derived and formulated in any number of ways, for example as described in U.S. Pat. No. 5,275,826, U.S. Pat. No. 5,554,389, U.S. Pat. No. 6,099,567, U.S. Pat. No. 6,375,989, and U.S. Pat. No. 6,379,710, U.S. Pat. No. 6,576,265, and to name a few types of extracellular matrices and how to make them. The extracellular matrix can be derived from any tissue in a mammal containing extracellular matrix, including (but not limited to) small intestine, bladder, liver, heart, lung, stomach, any soft tissue, enamel tissue, and the like. The extracellular matrix can be part of the matrix, or an entire set of multiple layers that make up the extracellular matrix. For example, the extracellular matrix can be just the submucosa, or just the basement membrane of a tissue, or it can be multiple layers that include the usually several layers of matrix that surround the environment of cells.

The matrix will be acellular, which can be achieved by standard techniques including treating the matrix with buffers and solutions that remove the cells from the matrix material. The matrix is mostly made up of extracellular matrix protein, with also some lipids and carbohydrates included. There is much discussion about the protein make-up of certain matrices, and indeed the protein make-up of the matrices derived from different tissues vary, however, for the purpose of this invention, all extracellular matrices will inhibit the abnormal cellular proliferation that is the target of the inventive methods disclosed herein. Extracellular matrix from enamel matrix has been described for inducing cell apoptosis in US patent publication 2005/0043216 to Hammarstrom et al., and as such enamel matrix is specifically excluded from the compositions and methods of this invention. Enamel matrix has also been described in U.S. Pat. No. 7,033,611 for use in allowing tissue to heal after tumor removal, particularly with breast tumor removal, and as such enamel matrix is specifically excluded from the methods and compositions of this invention. The inventions described in both the publication and patent are commonly owned, and the inventors appear to have failed to appreciate the breadth of the invention captured by this inventor: namely that any extracellular matrix can be used to fill a tumor cavity and facilitate cancer-free wound healing resulting in regeneration of lost tissue and reduced scar formation.

The extracellular matrix for the compositions and methods is from a mammalian source and can be any extracellular matrix from any tissue in a mammal. Mammals can include humans, horses, monkeys, cows, pigs, sheep, dogs, rabbits, rodents, and generally any otherwise healthy mammal. The extracellular matrices can be from any mammalian tissue having an extracellular matrix, particularly matrices that support soft tissue, and not hard tissue like enamel. Enamel matrices are excluded from the compositions of the invention. The extracellular matrices can be from for example, small intestine, liver, urinary bladder, stomach, pancreas, placenta, large intestine, heart, lung, kidney, and in general any tissue in the mammalian body. Fetal extracellular matrices can be used from any fetal organ of any mammal. It is also possible that other animals, including fish and birds may provide extracellular matrix of sufficient quality for use in the compositions. Additives can be mixed into the extracellular matrix. Additives can include such molecules as immunotherapeutic molecules, cells, anticancer agents, nucleic acids, peptides, polypeptides and proteins.

The extracellular matrix can be solid, semi-solid or liquid. The solid extracellular matrixes can be a sheet, a particulate, a small piece, patch, strip, pellet, plug, strand, weave, or any other form of solid extracellular matrix suited to the task of contacting the abnormal cells or the tissue at the resected tumor site in vivo. Preferred forms are sheets or particulate. Particulate can be made from drying sheets and breaking them up in to fine powder that can be stored, reconstituted, used as is, or delivered in a particular mode, for example by spray or dusting.

Extracellular matrix is a generic term for the proteinous material that exists outside of cells in tissues, supporting cells and cell-protein interactions, among many other functions. Extracellular matrix from epithelial tissues typically has a basement membrane layer, a submucosal layer, and a mucosal layer. Some tissues have an interstitial layer, lamina propria, tunica propria and other layers of matrix. Many of these layers are distinct for particular tissues, and some layers have synonymous terms in the same or different tissues. The invention contemplates any and all of these layers or divisions of matrix, either all together, or separately, or in various combinations to form the compositions of the extracellular matrix. It is generally believed that the submucosal layer is the most active and important of the extracellular matrix layers, and as such, preferred compositions and methods include at least a submucosa in the composition. Submucosa as derived from some mammalian tissues is described in U.S. Pat. No. 4,902,508, U.S. Pat. No. 5,281,422, U.S. Pat. No. 5,281,422, U.S. Pat. No. 5,275,826, U.S. Pat. No. 5,554,389, and other related US patents. Liquid extracts of these matrices and how to make them are described in U.S. Pat. No. 6,375,989 and U.S. Pat. No. 6,579,538, describing how to make liquid and semi-solid extracellular matrix compositions. All of these patents and any related commonly owned patents and patent applications with supportive disclosure are hereby incorporated by reference in their entirety. Generally to make the extracts of matrices, sheets are prepared from the tissues and lyophilized, and then broken up into a fine powder that can be reconstituted in saline or other suitable buffer at a desired concentration.

Accordingly, the particulate, emulsion or gel compositions can comprise extracellular matrix (ECM) and other materials as well. Optimally, the composition comprises only ECM, and the ECM been processed so as to retain key growth factors and other molecules and proteins so that when the ECM is placed in the body, the ECM remodels to become the host tissue with which it is in contact. Other materials added to the ECM could be, for example, a therapeutic agent, a drug, added proteins or added cells.

Natural ECM materials suitable for use with the present invention include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508, 4,956,178, and 5,275,826; urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710. See also U.S. Pat. No. 5,554, 389, U.S. Pat. No. 4,902,508, and U.S. Pat. No. 5,281,422. All of these patents and any related commonly owned patents and patent applications with supportive disclosure are hereby incorporated by reference in their entirety.

Although these particularly named extracellular matrices are known and have been isolated and used, there may be other mammalian tissues from which extracellular matrix can be isolated and prepared and as such would be suitable for the purposes of the invention. Extracellular matrix-like materials are also generally described in the article “From Cell-ECM Interactions to Tissue Engineering”, Rosso et al, Journal of Cellular Physiology 199, 174-180 (2004). Enamel matrices, which are the extracellular matrix in the tissue around forming teeth, are described in U.S. Pat. No. 7,033,611. The disclosures of all references cited herein are incorporated in their entirety by reference. Extracellular matrices from these tissues have been isolated, processed to retain key growth factors and structural molecules, and dried to become solid materials (sheets and particulates). Particulate forms can be rehydrated in a suitable buffer to become fluidized or emulsion or gel forms. Presently, these extracellular matrix articles and compositions are used for tissue grafting, wound healing, and tissue regenerative purposes.

The invention proposes use of these ECM articles and compositions and materials and forms for placement in the space remaining after a tumor excision as a surgical procedure for the purpose of reducing a likelihood of tumor recurrence in the at the site of tumor excision. The ECM articles and compositions and materials and forms are also proposed in order to regenerate the tissue lost from the organ or tissue during the tumor excision. In addition, the ECM articles and compositions and materials and forms are also proposed in order to reduce localized scarring that can result from the surgical wound created by the surgical procedure. All of these activities are premised on the activity that occurs when the extracellular matrix remodels to healthy new tissue, and that is that stem cells are recruited to the site.

The extracellular matrix can be in a fluid or liquid form, for example an emulsion or otherwise injectable solution. The extracellular matrix can be in a semi-solid form, for example a gel, foam, glue, paste, or other semi-solid form. The semi-solid forms may be injectable depending on their viscosity, but they will be applicable to the abnormally proliferating cells or the resected tumor space as is appropriate for their viscosity. For example, a large mouthed syringe may inject most gels or foams into the breast before or after placing the implant at the site. Topical creams can be spread onto the implant or the surrounding tissue or both. In addition, a spray foam may address depositing the extracellular matrix in the breast cavity or on the implant. The extracellular matrix can also be a solid, for example any solid form including a powder or particulate that can be sprayed or dusted in a region. The extracellular matrix contacts the breast tissue and is applied to the site as emulsified, injectable, foam, gel, liquid, glue, paste, small piece, patch, strip, pellet, plug, strand, weave, spray, paint, cream and any malleable form. The extracellular matrix can be a sheet that is wrapped around the implant, or stitched around it. Depending on the nature of the tissue and site small patches, plugs, strips, pellets, strands or some other such similar fragments or pieces of solid or semi-solid extracellular matrix material can be used effectively at certain sites with certain tissues, depending largely on the architecture of the tissue, and considering how best to introduce the estracellular matrix. The preferred form of the extracellular matrix will be that form, either liquid, semi-solid or solid that provides maximal contact of the matrix with the tissue or cells that are targeted. So, for example, a solution or emulsion or gel of extracellular matrix will fill a closed space and provide ample contact of the matrix with the tissue of the surrounding region.

The composition of the invention is a pharmaceutical composition comprising a therapeutically effective amount of mammalian extracellular matrix comprising extracellular matrix protein. The extracellular matrix is preferably from a non-malignant tissue source, thus, the tissue from which the extracellular matrix is derived is a healthy tissue in which, presumably, there are no cancer or precancer cells. Others have described using extracellular matrix from the tissue that generates teeth enamel in U.S. Pat. No. 7,033,611, so that the extracellular matrix compositions described herein are generally from a non-enamel source, being from any other tissue in the body, and thus being any other extracellular matrix material except what is called enamel tissue, that tissue growing adjacent to and fostering teeth enamel growth. At certain very high concentrations, the compositions of the invention can also include teeth enamel matrix, at concentrations of extracellular matrix protein greater than about 30 mg/ml. Otherwise, the extracellular matrix pharmaceutical compositions are semi-solid or liquid and comprise extracellular matrix from non-malignant non-enamel tissue comprising extracellular matrix proteins in a concentration greater than about 1 mg/ml, greater than about 2 mg/ml, greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 11 mg/ml, greater than about 12 mg/ml, greater than about 13 mg/ml, greater than about 14 mg/ml, greater than about 15 mg/ml, greater than about 16 mg/ml, greater than about 17 mg/ml, greater than about 18 mg/ml, greater than about 19 mg/ml, greater than about 20 mg/ml, greater than about 21 mg/ml, greater than about 22 mg/ml, greater than about 23 mg/ml, greater than about 24 mg/ml, greater than about 25 mg/ml, greater than about 26 mg/ml, greater than about 27 mg/ml, greater than about 28 mg/ml, greater than about 29 mg/ml, greater than about 30 mg/ml, greater than about 31 mg/ml, greater than about 32, mg/ml, greater than about 33 mg/ml, greater than about 34 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, and about 100 mg/ml. The extracellular matrix protein concentration of these pharmaceutical compositions comprising extracellular matrix from a non-malignant and non-enamel source can be in a range of extracellular matrix protein concentration greater than about 1 mg/ml to about 100 mg/ml.

By the term extracellular matrix protein is meant any protein found in extracellular matrix material in any tissue of any mammal that is structurally or functionally integral to the formation, maintenance and structure of the extracellular matrix. Many different types of proteins in different classes with different structures and functions fit into this category of extracellular matrix protein. Different tissues have different extracellular matrix protein, in different amounts. In addition, certain proteins are deemed to be part of the extracellular matrix in that they provide some integral function in the formation and maintenance of the matrix itself, including cell signaling and other messengering activities within the matrix scaffold.

A composition is also presented comprising a therapeutically effective amount of a solid mammalian extracellular matrix in a particulate or powder form, this pharmaceutical composition comprising extracellular matrix protein greater than about 75% by weight of the composition. Accordingly, the solid extracellular matrix composition comprises powder or particulate having total weight of the extracellular matrix protein in the composition greater than about 80%, greater than about 85%, greater than about 90%, greater than about 95%, and about 100% of the weight of the entire pharmaceutical composition. The solid composition can comprise in a range from greater than about 75% weight of extracellular matrix protein to about 100% of the weight of the pharmaceutical composition.

The pharmaceutical composition can comprise a therapeutically effective amount of semi-solid or liquid mammalian extracellular matrix comprising extracellular matrix in a concentration of extracellular matrix protein greater than about 30 mg/ml. Thus, the extracellular matrix protein concentration of the semi-solid or liquid pharmaceutical composition can comprise greater than about 31 mg/ml, greater than about 32, mg/ml, greater than about 33 mg/ml, greater than about 34 mg/ml, greater than about 35 mg/ml, greater than about 40 mg/ml, greater than about 45 mg/ml, greater than about 50 mg/ml, greater than about 55 mg/ml, greater than about 60 mg/ml, greater than about 65 mg/ml, greater than about 70 mg/ml, greater than about 75 mg/ml, greater than about 80 mg/ml, greater than about 85 mg/ml, greater than about 90 mg/ml, greater than about 95 mg/ml, and about 100 mg/ml.

The composition of the invention also includes a pharmaceutical composition comprising a therapeutically effective amount of a semi-solid or liquid mammalian extracellular matrix comprising extracellular matrix protein in an amount greater than 55% wet weight (w/w/) of the extracellular matrix protein. The amount of extracellular matrix protein can be thus, greater than 60% w/w, greater than 65% w/w, greater than 70% w/w, greater than 75% w/w, greater than 80% w/w, greater than 85% w/w, greater than 90% w/w, greater than 95% w/w, and about 100% w/w. The amount of extracellular matrix protein can be in a range from greater than 55% w/w to 100% w/w.

For the purposes of this invention, the term pharmaceutical in the phrase pharmaceutical compositions means that the compositions also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent” and “drug” are used, then, or when a particular active agent is specifically identified, it is to be understood that applicants intend to include the active agent per se as well as pharmaceutically acceptable, pharmacologically active salts, esters, amides, prodrugs, metabolites, analogs, etc.

By “pharmaceutically acceptable,” such as in the recitation of a “pharmaceutically acceptable carrier,” or a “pharmaceutically acceptable acid addition salt,” or “pharmaceutically acceptable salt” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative, refers to a derivative having the same type of pharmacological activity as the parent compound and approximately equivalent in degree.

When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA. Thus pharmaceutical compositions include an extracellular matrix component, or derivatives or active agents related to it, and also possibly additional pharmaceutical agents that complement, or otherwise contribute to the function of the composition as a whole, such as a carrier, exipient, or the like.

By an “effective” amount or a “therapeutically effective amount” of a pharmacologically active agent is meant a nontoxic but sufficient amount of the agent to provide the desired effect of facilitating reduced scarring in breast implant procedures. This amount is probably related to the size of the implant in the patient, and can be addressed with adjustments to the concentration or absolute amount of protein in the extracellular matrix composition, and to the volume of the composition that is added to the site of tumor excision.

The extracellular matrix can be formulated in a pharmaceutical composition for administration to the patient. The pharmaceutical composition can include excipients or active agents other than the extracellular matrix components. The pharmaceutical composition can be a liquid, a semi-solid or a solid, as described above.

The contacting step in which the tissue is contacted with a pharmaceutical composition comprising extracellular matrix can be accomplished in such a way that the amount of extracellular matrix per volume of space near the cell is greater than about 0.001 mg/cm². In fact the amount of extracellular matrix per volume of space near the cell can be in a range from greater than about 0.001 mg/cm² to about 50 mg/cm². Accordingly, the amount of extracellular matrix per volume can be greater than about 1 mg/cm², 5 mg/cm², 10 mg/cm², 15 mg/cm². 20 mg/cm², 25 mg/cm², 30 mg/cm², 35 mg/cm², 40 mg/cm², 45 mg/cm², and including about 50 mg/cm²,

Detection of developing carcinoma before tumor excision, recurring carcinoma after excision, or forming scar tissue (or lack thereof) after tumor excision followed by placement of ECM at the site of tumor excision can be accomplished by a novel technology called Magnetic Resonance Elastography (MRE). MRE can be used to determine the stiffness of inelasticity of a particular tissue. Where, for example, fat tissue has a kPa of 4, and healthy tissue has a kPa of 6, cancer (carcinoma) will often have a kPa in the realm of a value of 20. Scar tissue may have a kPa between that of healthy tissue and cancer, or perhaps a value stiffer than that of cancer. The absolute value of various scar tissues in the various tissues and organs of interest for the purposes of this invention can be determined by routine application of MRE to these tissues or organs in patients both having and not having carcinoma or scarring. Basic information on the technology called MRE can be found at Doyley et al. “Thresholds for detecting and characterizing focal lesions using steady-state MR elastography,” Medical Physics, 30(4):495-504, 2003.

Detection of infiltrating stem cells that have been recruited to a site of malignancy (e.g. where a tumor used to be and has now been removed) can be made by histology, showing precursor cells in the regenerating tissue where the exogenous mammalian extracellular matrix is placed in the patient. ECM can also be placed at the site of tumor without tumor removal to induce recruitment of adult stem cells to the site of malignancy. Alternatively, the tumor and some surrounding tissue can be removed and ECM placed at the site of tumor removal to recruit adult stem cells to the site in the process of regenerating lost tissue.

### EXPERIMENTAL

For these experiments 1-4, mice are selected for testing the invention. The genes of mice are similar to humans and so mice provide a suitable initial animal model for testing cancer treatments. Jackson laboratory has several strains of mice available that are appropriate for experiments involving the invention. Notably, several JAX® mice have been bred for increased tumor incidence: (JAXmice.jaxorg/models/cancer)

A/J 000646

BALB/cByJ (000651)

CBA/CaJ (000654)

In addition several strains have been developed with special specific propensities:

C57BL/6J-Apc^{MIN} (002020)—propensity to develop adenomas

FVB/N—TgN (MMTV neu) 202Mul (002376)—propensity towards mammary tumors

C57BL/6—TgN (TRAMP) 8247Ng (003135)—propensity to develop prostate tumors

### EXAMPLE 1

### 000654

As an initial experiment CBA/CaJ (000654) mice are selected. The 000654 mouse has an increased incidence of late onset mammary gland tumors, and also a propensity towards hematomas and lymphomas.

The CBA inbred strain is susceptible to tumor induction after a single subcutaneous injection of methyl cholanthrene. Accordingly, three female 000654 mice are ordered from Jackson Laboratories in Bar Harbor, Me. (JAX labs). All three mice receive an injection of methyl cholanthrene at 8 weeks in a single injection. The mammary glands of each mouse are identified and marked. All mammary glands are injected with sufficient ml of methyl cholanthrene to cause tumors to grow. The mice are observed for tumor development which is diagnosed by palpation. As the tumors develop, the three mice are treated as follows:

Mouse A—tumor removal, closure of the site and continued observation. The tumor tissue is retained for analysis. An aliquot of blood and lymph is extracted to test for evidence of metastasis.

Mouse B—tumor removal, placement of extracellular matrix emulsion at the site of excision and closure of the wound. The tumor tissue is retained for analysis. An aliquot of blood and lymph is extracted to test for evidence of metastasis.

Mouse C—partial tumor excision, leaving some cancer cells at the site, application of extracellular matrix emulsion at the site of excision and closure of the wound. The tumor tissue is retained for analysis. An aliquot of blood and lymph is extracted to test for evidence of metastasis.

Once a tumor is observed in a mouse, the lesion is surgically removed and the tumor tissue is analyzed to confirm the nature and stage of the cancer cells. An emulsion of extracellular matrix material is placed at the site of tumor removal and the incision is closed. The mouse is monitored for a recurrence of the lesion. If other tumors develop in other glands, those are also removed and the site is filled with an emulsified extracellular matrix material.

At 4 to 6 months the mice are sacrificed. The surgical sites of each site of excision are removed and analyzed for tumor cells, tissue quality, and presence of extracellular matrix. The sites are spread on a slide and photographed. The mouse blood and lymph are tested for evidence of metastasis.

### EXAMPLE 2

### A/J Mice

A/J mice have a propensity to develop lung tumors in response to carcinogens. A/J mice also have a high incidence of mammary adeno-carcinomas in multi-parous females. Four females are purchased and observed for development of either lung or mammary tumors. When tumors develop in the mice, the tumors are removed and the site injected with ECM and closed.

Mouse A tumor removed completely, ECM added, and site closed.

Mouse B tumor removed partially, ECM added and site closed.

Mouse C tumor not removed, ECM added and site closed.

Mouse D tumor not removed, no surgery. Tumor monitored for progression of growth.

### EXAMPLE 3

### 002020

C57BL/6J-Apc min is a strain of mouse that is highly susceptible to spontaneous intestinal adenoma formation. One hundred percent of C57BL/6J—Apc min heterozygous mice raised on a high fat diet develop in excess of 30 adenomas through the intestinal tract and most die by 120 days of age. A small number of female mice develop mammary tumors.

Mouse A—control—tumors allowed to develop unchecked.

Mouse B, C, D, E,—tumor development observed

After tumor development is established, two groups are created:

3 mice 1. surgical group+ECM after surgery

3 mice 2. surgical group+no ECM after surgery

As many tumors as develop in these mice and are detected are either removed and contacted with ECM, or simply removed. The mice are observed for overall survivability, cancer or cancer-free condition. Blood and lymph are tested for evidence of metastasis.

### EXAMPLE 4

### 02376

Mice homozygous for the MMTV/neu (rat) transgene are viable and fertile. Focal mammary tumors first appear at 4 months, with median incidence of 205 days. Both virgin and breeder mice develop tumors. Tumors arise as hyperplastic and displastic tumors. Seventy-two percent of tumor bearing mice that lived to 8 months or longer developed metastatic disease to lung.

Female mice are purchased; mice observed for mammary tumor development.

Mouse A control, no surgery, no ECM

Mouse B surgery plus ECM

Mouse C surgery—no ECM

Mouse D inject ECM at tumor site only (without opening the site).

Those mice that survive past 8 months, are observed for lung carcinoma:

Mouse B surgery+ECM at lungs, if lung tumor develops

Mouse C surgery+no ECM

Mouse D inject ECM at the tumor site only.

### EXAMPLE 5

### Actual Study with Nude Mice having SubQ Tumors

Three groups of nude mice from Charles Rivers laboratory were formed from 24 mice. MDA-MB-231 human breast cancer cells were received from ATCC and cultured to confluence for inoculation in the animals subcutaneously. Each mouse was inoculated beneath the skin in the right and left flanks. Two mice in each group using 1 million cells, two mice using 2.5 million cells, two mice using 5 million cells, and 2 mice using 10 million cells. One group was just cancer cells alone. The second group was matrigel™ plus cells, and the third group was extracellular matrix emulsion from small intestine submucosa with the cancer cells. As of the date of this filing of this patent application. Tumors were detected in the matrigel mice, at 5 million and 10 million cells. No tumors were detected in the extracellular matrix mice, and no tumors were yet detected in the cells only mice. Before complete conclusions can be drawn about the growth inhibitory potential for extracellular matrix in the presence of cancer cells, tumors will have to appear in the control animals. It is expected that within about 2 weeks tumors will be detected in the control animals. Following the completion of the study the mice will be sacrificed and the tumors will be analyzed histologically.

Criticism of this experiment includes primarily that in this experiment by design the tumor cell inhibition is being studied outside the context of epithelial cancer cells growing in epithelial tissue surrounded by its native extracellular matrix. The tumor cells are injected subcutaneously and therefore grow without a native epithelial matrix to support them. One precept of the invention is that abnormal epithelial cells inhibition will most optimally be demonstrated by contacting the abnormally proliferating cell as it is proliferating at the epithelium of origin. This can not be demonstrated in a subcutaneous model such as tested in this example. Whether inhibition of the subcutaneously implanted human breast cancer cells will occur with in the presence of the extracellular matrix remains to be seen as the data is not complete for these experiments, but the inventor believes that the most optimal demonstration of the efficacy and indeed true nature of the invention will be accomplished by placing a therapeutically effective amount of a composition comprising extracellular matrix at a site where the carcinoma cells are growing in the epithelium of origin. The inhibition of abnormal cell proliferation, and the inhibition of tumor regrowth after resection will be shown when the extracellular matrix is placed at this in vivo site in the actual context of an in situ carcinoma. Local epithelial tissue will be removed or disturbed with the removal or disturbance of abnormally proliferating cells at the site, and contact at this site with extracellular matrix from a mammal will demonstrate growth inhibition, and inhibition of tumor recurrence, as well as general healing of the damaged or diseased tissue.

It is hypothesized that the system may work best in the examples 1-4 that uses tumors generated at the site of origin for the study, so that the tumors can be studied and analyzed in contact with extracellular matrix in the actual context of the epithelial tissues. With these studies the extracellular matrix is facilitating healing and inhibition of abnormal proliferation in the context of epithelial tissue and it is thought by the inventor that this may be a more accurate assessment of the capabilities of the extracellular matrix composition in the animal afflicted with an abnormal cell proliferation disorder.

### EXAMPLE 6

### Prospective Application of Extracellular Matrix in Humans After Tumor Removal in Order to Recruit Adult Stem Cells to the Site of Malignancy

Surgical candidates are selected from a group of patients diagnosed with carcinoma in various organs or tissues, such as liver, lung, breast, pancreas, ovary, esophagous, skin, colon, prostate, or other organs or tissues in which carcinoma tumors can develop. Those patients deemed eligible for treatment by surgical removal of their tumors and surrounding tissue are prepared for surgery. Removal of the tumor and surrounding tissue is followed by placement of an extracellular matrix material at the site of tumor removal. The form of extracellular matrix material that is used can be sheet matrix, particulate matrix, emulsion, fluidized matrix, or other appropriate form of extracellular matrix. The patient is closed and recovery is monitored non-invasively by MRE and periodic circulating marker analysis. The MRE technology is used to detect recurring carcinoma, scar tissue formation (or lack thereof), and new regenerating tissue at the site of tumor removal. Core biopsy (followed by histological and pathological analysis) is used to detect recruited stem cells to the new tissue.

All references cited are incorporated in their entirety. Although the foregoing invention has been described in detail for purposes of clarity of understanding, it will be obvious that certain modifications may be practiced within the scope of the appended claims.

What is claimed is:

1. A method of recruiting an endogenous stem cell to a site of abnormally proliferating cells in mammalian tissue comprising:
a) locating a site of an abnormally proliferating cell in a tissue type of a mammal,
b) contacting said site with a composition comprising exogenous native mammalian extracellular matrix, and
c) detecting recruitment of an endogenous stem cell to said site.

2. The method of claim 1, wherein said abnormally proliferating cell is an epithelial cell.

3. The method of claim 1, wherein said tissue type is epithelial tissue.

4. The method of claim 1, wherein said extracellular matrix is liquid or semi-solid.

5. The method of claim 1, wherein said extracellular matrix is a particulate.

6. The method of claim 1, whererin said extracellular matrix is a sheet.

7. The method of claim 1, wherein said extracellular matrix is porcine, bovine, or human.

8. The method of claim 1, wherein said extracellular matrix is small intestine submucosa, liver basement membrane, urinary bladder submucosa, or stomach submucosa.

9. The method of claim 1, wherein said abnormally proliferating cell is malignant.

10. The method of claim 1, further comprising a step of disturbing said abnormally proliferating cell at said site before contacting said site with said composition.

11. The method of claim 10, wherein said disturbing step comprises removal of all or part of said site of abnormally proliferating cells.

12. The method of claim 1, wherein said stem cell is an adult stem cell.

13. The method of claim 1, wherein said mammalian tissue is selected from the group consisting of pancreatic tissue, liver tissue, breast tissue, ovarian tissue, and lung tissue.
